(19) United States
(12) Patent Application Publication
(10) Pub. No.: US 2008/0299050 A1
(43) Pub. Date: Dec. 4, 2008

(54) TOPICAL THERAPIES FOR ORAL MUCOSITIS AND OTHER CONDITIONS

(75) Inventors: Jonathan David Bortz, St. Louis, MO (US); R. Saul Levinson, Chesterfield, MO (US); Jisheng Ge, Chesterfield, MO (US); Jeremy Donald Wang, Frontenac, MO (US)

Correspondence Address: KV PHARMACEUTICAL COMPANY, 4080B WEDGEWAY COURT, EARTH CITY, MO, 63045, US

(73) Assignee: DrugTech Corporation, Wilmington, DE (US)

(21) Appl. No.: 11/865,476

(22) Filed: Oct. 1, 2007

Related U.S. Application Data
(60) Provisional application No. 60/828,319, filed on Oct. 5, 2006.

(51) Int. Cl. A61K 8/18; A61K 31/4166; A61K 31/485; A61P 25/04

(52) U.S. Cl. 424/49; 514/398; 514/282

(57) ABSTRACT

A method for treating oral mucositis in a subject comprises topically administering to an oral mucosal surface of the subject phenyloin or a pharmaceutically acceptable salt thereof in an amount effective to inhibit mucosal degeneration or promote mucosal regeneration, and optionally an analgesic agent in an amount effective, in combination with the phenyloin or salt thereof, to reduce pain associated with the oral mucositis. The phenyloin or salt thereof and optionally the analgesic agent can be administered in a pharmaceutical composition comprising an excipient vehicle suitable for intraoral administration, said composition being bioadhesive to an oral mucosal surface, for example having at least one non-lipoidal internal phase and at least one lipoidal external phase that is bioadhesive to said surface.

This application claims the benefit of U.S. provisional application Ser. No. 60/828,319, filed on Oct. 5, 2006, the entire disclosure of which is incorporated herein by reference.

### FIELD OF THE INVENTION

The present invention relates to methods for treating oral mucositis, methods for treating painful skin lesions, and pharmaceutical compositions useful in such methods. The invention further relates to cancer treatment regimens.

### BACKGROUND OF THE INVENTION

Oral mucositis is a debilitating inflammatory disease of the oral mucosa, often manifested as erythema and painful ulcerative lesions of the mouth, in some cases also affecting the throat (oropharyngeal mucositis). Oral mucositis is a well-known complication of cancer therapies involving radiation therapy and/or chemotherapy, occurring in about 40% of patients receiving such therapies. Best Practice 2(3) (1998), available at http://www.oralcancerfoundation.org/dental/pdf/mucositis.pdf.

Further, approximately 70-80% of patients with hematological malignancies undergoing hematopoietic stem cell transplantation suffer from oral mucositis. See, for example, Amgen product information on Kepivance® (palifermin), available for example at http://www.kepivance.com.

Oral mucositis is defined by the National Cancer Institute (NCI) as inflammation of oral mucosa resulting from chemotherapeutic agents or ionizing radiation, and as a type of stomatitis, which refers generally to inflammation of oral tissue, including mucosa, dentition/periapices and periodontium (see http://www.cancer.gov/cancertopics/pdq/supportivecare/oralcomplications/HealthProfessional/page5).

Oral mucositis can result from systemic effects of cytotoxic chemotherapy agents and from local effects of radiation therapy. Typically oral mucositis develops within 7 to 14 days after initiation of chemotherapy or radiation therapy. The disease runs a course in which five phases have been recognized (see http://www.kepivance.com).
Phase 1 (initiation): radiation or chemotherapy causes DNA damage in basal epithelial cells and generates reactive oxygen species (ROS), which further damage cells and blood vessels in the submucosa.
Phase 2 (signaling): chemotherapy, radiation and ROS induce apoptosis and upregulate inflammatory cytokines in cells.
Phase 3 (amplification): inflammatory cytokines produce further tissue damage, amplifying signaling cascades and the injury process.
Phase 4 (ulceration): loss of mucosal integrity produces extremely painful lesions, providing portals of entry for bacteria, viruses and fungi.
Phase 5 (healing): proliferation, differentiation and migration of epithelial cells to restore the integrity of the mucosa.

Common toxicity criteria have been established by NCI for grading severity of oral mucositis (http://ctep.cancer.gov/forms/CTCv20_{—}4-30-992.pdf).
NCI Grade 0: none.
NCI Grade 1: painless ulcers, erythema or mild soreness in the absence of lesions.
NCI Grade 2: painful erythema, edema or ulcers, but the patient can eat or swallow.
NCI Grade 3: painful erythema, edema or ulcers requiring intravenous hydration.

NCI Grade 4: severe ulceration or the patient requires parenteral or enteral nutritional support or prophylactic intubation.

Criteria established by the World Health Organization (WHO) are generally similar (see http://www.kepivance.com).

WHO Grade 0: none.

WHO Grade 1: soreness±erythema.

WHO Grade 2: erythema, ulcers; the patient can swallow a solid diet.

WHO Grade 3: ulcers, extensive erythema; the patient cannot swallow a solid diet.

WHO Grade 4: mucositis to the extent that alimentation is not possible.

Oral mucositis has become a common and often treatment-limiting side effect of therapy for cancers, particularly but not exclusively for cancers of the head and neck, especially where the therapy includes radiation. Few interventions have proven effective for treatment (including prophylaxis) of oral mucositis. Currently, there are no universally accepted treatment protocols for prevention of oral mucositis in patients receiving chemotherapy and/or radiation therapy. Palliative care is standard in management of oral mucositis, and can include
bland rinses, for example with 0.9% saline solution, sodium bicarbonate solution or 0.9% saline/sodium bicarbonate solution;
topical anesthetics, for example viscous formulations, ointments and sprays comprising lidocaine; sprays or gels comprising benzocaine; 0.5% or 1% dyclonine hydrochloride; or diphenhydramine solution;
mucosal coating agents, for example aluminum hydroxide suspension (e.g., Amphojel®); bismuth subsalicylate suspension (e.g., Kaopectate®); products containing film-forming agents (e.g., Zilactin-B®); cyanoacrylate mucoadherent film; or Gelclair® bioadherent oral gel;
analgesics, for example benzydamine hydrochloride topical rinse or opioid drugs administered orally, intravenously (e.g., bolus, continuous infusion, patient-controlled analgesia), transdermally by patch, or transmucosally;
growth factor, for example keratinocyte growth factor 1 (e.g., palifermin, specifically to decrease incidence and duration of severe oral mucositis in patients undergoing high-dose chemotherapy with or without radiation therapy followed by bone marrow transplant for hematological cancers).
   (See NCI information on oral mucositis, available for example at http://www.cancer.gov/cancertopics/pdq/supportivecare/oralcomplications/HealthProfessional/page5.)

Also helpful are good oral hygiene practices and cryotherapy (for example by means of ice chips placed in the mouth). Various mouthwashes have also been used. Most institutions have their own version of a “magic mouthwash” which is typically a preparation containing lidocaine, diphenhydramine and a coating agent such as aluminum hydroxide.

Epstein & Schubert (2003) Oncology 17(12):1767-1779 review current and developing therapies for oral mucositis. Current care is said to be essentially palliative and to include appropriate oral hygiene, non-irritating diet and oral care products, topical palliative mouth rinses, topical anesthetics, and opioid analgesics. Systemic analgesics are said to be the mainstay of pain management but topical approaches are reported to be under investigation. The authors state that literature supports use of benzydamine for prophylaxis of mucositis caused by conventional fractionated head and neck radiotherapy, and cryotherapy for use with short-half-life stomatotoxic chemotherapy such as bolus fluorouracil. Potential use of biologic response modifiers and growth factors, including topical and systemic delivery of epithelial growth factors and agents, is also mentioned.

Cerchietti et al. (2002) Cancer 95(10):2230-2236 reported comparison of a morphine mouthwash with a “magic mouthwash” containing lidocaine, diphenhydramine and magnesium aluminum hydroxide, for topical treatment of pain associated with oral mucositis in patients receiving chemoradiotherapy for head and neck cancer. Both duration and intensity of pain, as well as duration of severe functional impairment, were reported to be lower in the morphine-treated patients than in those receiving “magic mouthwash”.

Cerchietti et al. (2003) Pain 105(1-2):265-273 reported a pilot study in which oral mucositis patients received oral rinses of 15 ml of a 0.1% or 0.2% morphine solution 2- or 3-hourly. No systemic absorption of morphine was detected.

International Patent Publication No. WO 02/41837 mentions methods for treating mucositis comprising contacting a mucosal site, for example an oral mucosal site, with a composition comprising a pharmaceutical substance and a biocompatible polymer (e.g., poloxamer 407) that adheres to the mucosal site. Among pharmaceutical substances mentioned as being useful are antioxidants, antibacterials, anti-inflammatories, anesthetics, analgesics, proteins, peptides and cytokines.

U.S. Pat. No. 6,509,028 to Williams et al. describes compositions comprising a mucoadhesive (e.g., poloxamer 407), a local anesthetic (e.g., lidocaine) and an opioid (e.g., morphine or a pharmaceutically acceptable salt thereof, such as morphine sulfate pentahydrate. Such compositions are said to be useful for topical administration, for example as a sprayable liquid, to mucosa of the buccal or nasal cavity to induce local anesthesia in the mucosa, for example in cases of oral mucositis.

U.S. Pat. No. 5,554,380 to Cuca et al provides a bioadherent orally ingestible drug delivery system comprising about 25% to about 75% by volume of an internal hydrophilic phase and about 25% to about 75% by volume of an external hydrophobic phase that comprises an emulsifier, a glyceride ester and a wax material. Any stable drug or combination thereof is said to be employable in such a system; among a long list of categories of drugs mentioned can be found anticonvulsants and antipyretic and analgesic agents. The drug delivery system can be an orally usable emulsion or suspension in solid or semi-solid form, or an emulsion delivered in a carrier vehicle such as a chewing gum or confectionery composition. A property said to be common to all formulations embraced is that they coat and adhere to mucosal membranes of the mouth, pharynx, esophagus and/or gastrointestinal (GI) tract for extended periods of time. The patent states: “A distinct advantage of using a bioadhesive substrate on the oral and esophageal mucosa is to effect the systemic circulation of sufficient active agents”. Among uses of the drug delivery system is a “method for treating an oral or esophageal disorder”. Oral disorders mentioned include stomatitis.

U.S. Pat. No. 5,858,391 to Cuca et al. provides a bioadherent orally ingestible system comprising about 75% to about 99% by volume of an internal hydrophilic phase and about 1% to about 25% by volume of an external hydrophobic phase that comprises a hydrophobic oil and an emulsifier having an HLB (hydrophilic/lipophilic balance) value less than about 10. The system can be a liquid or semi-liquid emulsion or suspension, or an emulsion delivered in a carrier vehicle such as a chewing gum or confectionery composition. A property said to be common to all formulations embraced is that they coat and adhere to mucosal membranes of the mouth, pharynx, esophagus and/or GI tract for extended periods of time. The patent states: “A distinct advantage of using a bioadhesive substrate on the oral and esophageal mucosa is to effect the systemic circulation of sufficient active agents”. Among an extensive list of specific examples of active agents mentioned is morphine. Anticonvulsants are also mentioned.

It has long been known (Kimball & Horan (1939) Ann. Internal Med. 13(5):787-793) that the anticonvulsant drug phenyloin sodium, when administered systemically, can produce gingival hyperplasia as a side effect.

Shapiro (1958) Exp. Med. Surg. 16:41-53 reported that phenyloin sodium, administered by mouth for up to 8 weeks prior to gingival excision, greatly enhanced gingival wound healing. A marked acceleration of fibroblastic activity, clot organization and epithelial proliferation was said to occur.

Goebel (1972) J. Oral Surg. 30:191-195 reported a clinical study comparing oral healing in patients taking phenyloin sodium (200-300 mg daily) versus patients receiving chlorpromazine or no drug.

Moy et al. (1985) Arch. Dermatol. 121:79-83 reported that phenyloin (25 μg/ml) modulates cell proliferation in human slin fibroblast cultures in a way that is both concentration- and time-dependent. Cell proliferation was enhanced by phenyloin at low concentration (5 μg/ml) and short incubation time (3 h) but at higher concentration (25 or 50 μg/ml) or with longer incubation time (25 h or 48 h) this effect was reduced or reversed.

Modaghegh et al. (1989) Int. J. Dermatol. 28(5):347-350 applied a thin layer of phenyloin sodium powder topically to war and non-war wounds to enhance healing.

Arnstead et al. (1996) Ann. Pharmacother. 30:768-775 reported that, even with a very large dose of phenyloin applied topically to a pressure ulcer, little systemic absorption of phenyloin occurred.

Rhodes et al. (2001) Ann. Pharmacother. 35:675-681 prepared a topical phenyloin suspension by adding the contents of a 100 mg phenyloin capsule to 5 ml of sterile 0.9% saline solution. Sterile gauze was soaked in the suspension and placed over a decubitus ulcer. More rapid ulcer healing was reported in patients receiving topical phenyloin than in those receiving DuoDerm® hydrocolloid dressings or triple antibiotic ointment applications.

Oral mucositis, particularly when severe, has a major impact on daily functioning, well-being and quality of life of a subject. It can interfere with normal oral function, including speech and oral intake of food. It can also compromise a patient's ability to tolerate planned cancer therapy, resulting in missed doses or dose reductions, and can thereby lead to a less successful outcome of such therapy, for example greater likelihood of recurrence of the cancer, shorter remission, or increased mortality. Oral mucositis negatively affects other health outcomes as well, for example increasing risk of opportunistic secondary infections and mortality due to sepsis. Therefore, new therapies are needed to prevent and treat oral mucositis.

### SUMMARY OF THE INVENTION

There is now provided a method for treating oral mucositis in a subject, comprising topically administering phenyloin or a pharmaceutically acceptable salt thereof to an oral mucosal surface of the subject in an amount effective to inhibit mucosal degeneration or promote mucosal regeneration.

There is further provided such a method, further comprising topically administering to the oral mucosal surface an analgesic agent, comprising for example morphine and/or a pharmaceutically effective salt thereof, in an amount effective, in combination with the phenyloin or salt thereof, to reduce pain associated with the oral mucositis.

There is still further provided a therapeutic combination comprising phenyloin and an analgesic agent, each in a pharmaceutical composition adapted for topical administration to a biological surface, for example an oral mucosal surface, and bioadhesive thereto. The combination can take a form of separate compositions, respectively comprising the phenyloin and the analgesic agent, or of a single composition comprising both the phenyloin and the analgesic agent.

There is still further provided a pharmaceutical composition comprising phenyloin or a pharmaceutically acceptable salt thereof in an excipient vehicle suitable for intraoral administration, the composition being bioadhesive to a biological surface, for example an oral mucosal surface.

There is still further provided such a composition, further comprising an analgesic agent, comprising for example morphine and/or a pharmaceutically effective salt thereof.

Illustratively, such compositions have at least one non-lipoidal internal phase and at least one lipoidal external phase that is bioadhesive to the oral mucosal surface.

There is still further provided a method for relieving pain from oral mucositis associated with cancer therapy in a subject, comprising administering to an oral mucosal surface of the subject a pharmaceutical composition comprising an analgesic agent that comprises morphine and/or a pharmaceutically acceptable salt thereof in an excipient vehicle suitable for intraoral administration, the composition having at least one non-lipoidal internal phase and at least one lipoidal external phase that is bioadhesive to the oral mucosal surface.

There is still further provided a cancer treatment regimen comprising (a) administering to a cancer patient radiotherapy and/or chemotherapy at a level sufficient to result in oral mucositis, (b) topically administering phenyloin or a pharmaceutically effective salt thereof to an oral mucosal surface of the patient in an amount effective to inhibit mucosal degeneration or promote mucosal regeneration, and optionally (c) topically administering to the oral mucosal surface an analgesic agent, comprising for example morphine and/or a pharmaceutically acceptable salt thereof, in an amount effective, in combination with the phenyloin or salt thereof, to reduce pain associated with the oral mucositis.

There is still further provided a cancer treatment regimen comprising administering to a cancer patient radiotherapy and/or chemotherapy at a level sufficient to result in oral mucositis, and topically administering to an oral mucosal surface a pharmaceutical composition that comprises an analgesic agent comprising morphine and/or a pharmaceutically acceptable salt thereof, in an excipient vehicle suitable for intraoral administration, the composition having at least one non-lipoidal internal phase and at least one lipoidal external phase that is bioadhesive to the mucosal surface.

There is still further provided a method for treating a painful skin lesion, comprising topically administering to the lesion and/or an area of skin adjacent thereto a composition comprising phenyloin or a pharmaceutically acceptable salt thereof in an amount effective to promote healing of the lesion and, optionally, an analgesic agent, comprising for example morphine and/or a pharmaceutically acceptable salt thereof, in an amount effective to reduce pain associated with the lesion, the composition having at least one non-lipoidal internal phase and at least one lipoidal external phase that is bioadhesive to a skin surface.

Additional embodiments are described in the detailed description that follows.

### DETAILED DESCRIPTION

In a first aspect of the invention, a method for treating oral mucositis in a subject comprises topically administering phenyloin or a pharmaceutically acceptable salt thereof to an oral mucosal surface of the subject in an amount effective to inhibit mucosal degeneration and/or promote mucosal regeneration.

The terms “treat”, “treating” and “treatment” herein will be understood, except where the context demands otherwise, to embrace prophylactic administration to a subject not yet presenting symptoms of the condition specified, in the present aspect oral mucositis, but at risk of developing the condition, as well as administration to a subject already having the condition. Treatment can address an underlying cause of the condition and/or can be palliative, i.e., act to reduce or relieve symptoms, especially (in the present case of oral mucositis) symptoms such as pain, dry mouth and functional impairment, e.g., of eating, drinking, swallowing, speech or sense of taste, that cause distress to the subject.

The term “oral mucositis” herein will be understood to embrace oropharyngeal mucositis and, consistent with the NCI definition cited above, is distinguished from the more general term “stomatitis”, which includes inflammatory conditions of any oral tissue, not limited to the oral mucosa. Chiefly, oral mucositis of interest herein is associated with cancer therapy that includes chemotherapy and/or radiation therapy.

In some embodiments, the method of the first aspect of the invention is used to treat severe oral mucositis. By “severe oral mucositis” herein is meant oral mucositis of grade 3 or grade 4 according to NCI or WHO classification.

The subject herein can be of any animal, particularly mammalian, e.g., primate, species, but most typically is a human patient having or at risk of developing the condition specified, in the present aspect oral mucositis. In one embodiment the subject is immunocompromised, for example by disease or as a result of therapeutic intervention. In another embodiment, the subject has received, is receiving or later receives cancer therapy comprising chemotherapy, radiation therapy or a combination thereof, such cancer therapy being associated with risk or incidence of oral mucositis.

Phenyloin (5,5-diphenyl-2,4-imidazolidinedione), in older literature referred to as diphenylhydantoin, is an anticonvulsant and anti-epileptic drug sold, for example, as Dilantin® and having the formula

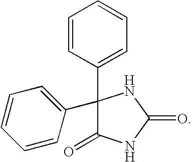

Pharmaceutically acceptable salts of phenyloin include the ammonium and alkali metal salts, more particularly the sodium salt. Phenyloin sodium is a water-soluble form of the drug and regenerates phenyloin upon dissociation in presence of an acid. It is sold, for example, as Epanutin®.

Unless the context demands otherwise, the term “phenyloin” herein will be understood to embrace pharmaceutically acceptable salts such as phenyloin sodium.

According to the present invention, phenyloin has the hitherto unrecognized property of inhibiting mucosal degeneration and/or enhancing mucosal regeneration in a subject having oral mucositis or at risk of developing oral mucositis, for example as a side effect of cancer therapy. It is believed, without being bound by theory, that this property arises from stimulation of endothelial hyperplasia, leading to more rapid replacement of mucosal tissues lost or destroyed as a result of the mucositis.

The phenyloin is administered topically to an oral mucosal surface, for example a surface of an oral mucous membrane that is affected by mucositis, or that is at risk of developing mucositis, for example as a result of ongoing or planned chemotherapy and/or radiation therapy. Topical administration herein includes directed placement of an agent, according to the first aspect of the invention phenyloin, on a target area of the oral (including pharyngeal) mucosa, for example by means of an oropharyngeal spray, a paste, gel or ointment, or a medicated wad or dressing. Topical administration herein further includes methods of contacting the oral (including pharyngeal) mucosa with the agent that do not necessarily involve directed placement, but that typically engage orofacial musculature, for example of the jaws, cheeks, tongue and throat, to distribute the agent within the oropharyngeal cavity. For example, swishing or gargling action, as with a liquid mouthwash or “swish and swallow” preparation, masticatory action, as with a soft chew or chewing gum preparation, or sucking or drawing action, as with a lozenge, can all be effective in bringing the agent into close contact with mucosal surfaces in the mouth and throat. Topical administration herein still further includes passive methods of applying the agent to a mucosal surface such as by simple dissolution or disintegration of, or release of the agent from, a solid dosage form such as a buccal or sublingual tablet or a quick-melt preparation.

Phenyloin, according to the first aspect of the invention, is administered in an amount effective to inhibit degeneration of the oral mucosa and/or, especially in cases of severe oral mucositis, to promote regeneration of the oral mucosa. Inhibition of mucosal degeneration can take the form of a slowing, cessation or partial to complete prevention of such degeneration. Promotion of mucosal regeneration can take the form of an increased rate or degree of healing of an inflamed, ulcerated or otherwise damaged mucosal membrane.

The amount of phenyloin effective to inhibit mucosal degeneration or promote mucosal regeneration depends on a number of factors, including the phase and severity of the oral mucositis and the phase of a cancer therapy cycle during which the phenyloin is to be administered. A physician of ordinary skill can determine a suitable amount to administer based on the present disclosure and on experience with a particular patient. In general, however, a suitable phenyloin dose will typically be found to be about 5 to about 500 mg/day, for example about 10 to about 400 mg/day, or about 25 to about 200 mg/day. In vitro studies have shown that different concentrations of phenyloin have different effects on fibroblast proliferation. The daily dose can be administered in a single application or split into a plurality of applications, for example at intervals of about 2 to about 12 hours. As disclosed hereinbelow, various embodiments of the present invention provide compositions having mucosal retention and drug release properties suited to a range of administration frequencies from 1 to about 8 times per day, for example 1 to about 6 times per day, or once or twice per day.

The phenyloin can be administered as straight active agent, for example in powder form, but for most purposes it will be found preferable to administer the phenyloin in a pharmaceutical composition that is adapted for intraoral administration. “Intraoral” herein refers to administration wherein a composition is placed in the oral cavity and is not immediately swallowed, so that release of an active agent from the composition can occur at least in part in the oral cavity. The active agent thus becomes available topically to the oral mucosa. Intraoral is thus distinguished from peroral administration, which typically leads to absorption in the lower GI tract.

Many examples are known of compositions that are adapted for intraoral administration. Illustratively and without limitation, such compositions include mouthwashes, swish and swallow liquids, viscous liquids (including solutions, suspensions and emulsions), gels, ointments, pastes, powders, reconstituted powders, films, chews, lozenges, troches, candies, lollipops, popsicles, chewing gums, sublingual and buccal tablets, quick-dissolve and quick-melt tablets, medicated wads and dressings, dentifrices and oropharyngeal sprays.

Depending on the type of intraoral composition and the dosage to be administered, the concentration of phenyloin therein can vary over a wide range. Concentration of the active agent in a sublingual tablet, for example, will typically be greater than in a lozenge or gel, which in turn will typically be greater than in a mouthwash. In general, the greater the volume of the composition that can conveniently be administered, the lower is the concentration of active agent needed in the composition. For each administration, a volume of about 0.5 to about 25 ml of an intraoral composition is typically administered, although lower and higher volumes can be suitable in particular situations. In a low-volume (about 0.5 to about 2 ml) composition, concentration of phenyloin can illustratively be about 10 to about 400 mg/ml, e.g., about 25 to about 200 mg/ml or about 50 to about 100 mg/ml; in a medium-volume (about 2 ml to about 15 ml) composition, illustratively about 1 to about 100 mg/ml, e.g., about 3 to about 50 mg/ml or about 10 to about 20 mg/ml; and in a high-volume (about 15 ml to about 25 ml) composition, illustratively about 1 to about 30 mg/ml, e.g., about 1 to about 20 mg/ml or about 2 to about 10 mg/ml.

In some embodiments of the method of the first aspect of the invention, the intraoral composition administered, or an ingredient or combination of ingredients therein, has physical properties that render it bioadhesive to the oral mucosal surface. Upon direct placement on a mucosal surface, or upon distribution within the oral cavity as described above, bioadherence of the composition to the mucosal surface typically results in firm contact and retention of the composition on the surface for a period of time that can be about 0.5 to about 24 hours or even longer, illustratively about 2 to about 24 hours, or about 2 to about 8 hours.

In this regard, it is noted that, especially in severe oral mucositis, the mucous membranes can be so degraded as not to have the physical properties of a healthy mucosal surface; thus, it will be understood that the term “bioadhesive to the oral mucosal surface” encompasses compositions that are bioadhesive to such a degraded mucosal surface. Not all compositions described in the art as “mucoadhesive” necessarily provide bioadherence appropriate to the present method.

Formulation systems providing bioadhesive properties are well known in the art and any such system can, in principle, be used herein. For example, bioadhesive polymers such as poloxamers, notably poloxamer 407 (available for example as Pluronic® F-127), can be incorporated in a composition.

In various embodiments, the composition has at least one non-lipoidal or hydrophilic internal phase and at least one lipoidal or hydrophobic external phase that is bioadhesive to the mucosal surface, as more fully described hereinbelow.

Of particular usefulness in the method of the first aspect are compositions exhibiting both bioadherence to the oral mucosal surface and controlled release, more especially sustained release, of phenyloin from the composition to the mucosa. Slow and sustained release of phenyloin is believed to result in maintenance of a therapeutically effective concentration of phenyloin in the mucosal and submucosal tissues, whereas an immediate or bolus release can lead, at least temporarily, to excessively high concentrations in the tissues that can be undesirable in one or both of the following ways:
at high concentrations, the growth-promoting, thus mucosal regeneration and healing, properties of phenyloin can be reduced or even reversed;
high local concentrations can increase the tendency for systemic uptake, resulting in the phenyloin being removed from the local site where its effect is desired to other parts of the body where it may produce unwanted side effects.

In one embodiment, the phenyloin is administered intraorally in a composition wherein bioadherence to the oral mucosal surface is sufficient to provide a retention period as described above, and wherein release of the phenyloin is sustained for most (i.e. more than about half) to all of the retention period. This can be accomplished, for example, by use of a composition having at least one non-lipoidal internal phase and at least one lipoidal external phase as described hereinbelow.

Optionally, the method of the first aspect of the invention further comprises administering a second agent topically to the oral mucosal surface. The second agent can co-act with the phenyloin to inhibit mucosal degeneration and/or promote mucosal regeneration. Alternatively or in addition, the second agent can have a palliative effect and/or can treat a secondary condition accompanying the oral mucositis, for example a microbial infection. The second agent can comprise a therapeutic agent and/or a nutritional agent such as vitamin E, vitamin C, vitamin A or glutamine. The second agent can be administered on a time schedule independent of or coordinated with the time schedule for phenyloin administration; for example, administration times, frequencies and compositions used can each be the same or different. In some embodiments, the phenyloin and the second agent are administered in separate intraoral compositions. In other embodiments, the phenyloin and the second agent are coformulated in a single intraoral composition which is administered topically to the oral mucosal surface.

A therapeutic agent useful in combination with phenyloin for topical administration in treatment of oral mucositis can illustratively be selected from analgesics, biologic response modifiers, antihistaminics, antimicrobials and antiseptics. Combinations of two or more agents from one or more of these classes can also be useful.

A presently preferred method of the first aspect of the invention comprises (a) topically administering phenyloin to the oral mucosal surface in an amount effective to inhibit mucosal degeneration or promote mucosal regeneration, and (b) topically administering to the oral mucosal surface an analgesic agent in an amount effective, in combination with the phenyloin, to reduce pain associated with the oral mucositis.

The analgesic agent can be selected from anesthetics, opioids and non-opioid analgesics. Combinations of two or more agents from one or more of these classes can also be useful.

In a more particular embodiment, the analgesic agent comprises an opioid. The opioid can illustratively comprise at least one agent selected from alfentanil, alphaprodine, anileridine, benzylmorphine, bezitramide, buprenorphine, butorphanol, clonitazene, codeine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, hydrocodone, hydromorphone, hydroxypethidine, isomethadone, ketobemidone, levorphanol, lofentanil, meperidine, meptazinol, metazocine, methadone, metopon, morphine, myrophine, nalbuphine, narceine, nicomorphine, norlevorphanol, normethadone, normorphine, norpipanone, opium, oxycodone, oxymorphone, papaveretum, pentazocine, phenadoxone, phenazocine, phenoperidine, piminodine, piritramide, proheptazine, promedol, propiram, propoxyphene, remifentanil, sufentanil, tilidine, and pharmaceutically acceptable salts, esters, prodrugs and enantiomers thereof.

Illustratively, the opioid comprises morphine and/or a pharmaceutically acceptable salt thereof, for example morphine sulfate or morphine hydrochloride.

Unless the context demands otherwise, the term “morphine” herein will be understood to embrace pharmaceutically acceptable salts such as morphine sulfate. It will further be understood that opioids other than morphine can be substituted if desired.

Morphine can be administered topically at any suitable dose providing relief of pain without provoking unacceptable adverse side effects. In most cases a morphine dose of about 5 to about 200 mg/day, for example about 10 to about 100 mg/day, about 20 to about 80 mg/day or about 30 to about 60 mg/day, will be found suitable. In various particular embodiments, the morphine dose is about 5 to about 10 mg/day, about 7.5 to about 15 mg/day, about 10 to about 25 mg/day, about 20 to about 40 mg/day, about 30 to about 60 mg/day, about 50 to about 100 mg/day, about 75 to about 150 mg/day or about 100 to about 200 mg/day.

As in the case of phenyloin, morphine is typically administered in a pharmaceutical composition that is adapted for intraoral administration, for example a mouthwash, swish and swallow liquid, gel, ointment, paste, powder, reconstituted powder, film, chew, lozenge, troche, candy, lollipop, popsicle, chewing gum, sublingual or buccal tablet, quick-dissolve or quick-melt tablet, medicated wad or dressing, dentifrice or oropharyngeal spray.

Depending on the type of intraoral composition and the dosage to be administered, the concentration of morphine therein can vary over a wide range. In a low-volume (about 0.5 to about 2 ml) composition, concentration of morphine can illustratively be about 5 to about 200 mg/ml, e.g., about 10 to about 100 mg/ml or about 20 to about 50 mg/ml; in a medium-volume (about 2 ml to about 15 ml) composition, illustratively about 1 to about 50 mg/ml, e.g., about 5 to about 30 mg/ml or about 10 to about 20 mg/ml; and in a high-volume (about 15 ml to about 25 ml) composition, illustratively about 0.5 to about 10 mg/ml, e.g., about 0.6 to about 6 mg/ml or about 1 to about 4 mg/ml.

The composition comprising morphine can, like the phenyloin-containing composition, be bioadhesive to the oral mucosal surface, exhibiting a retention time thereon of about 0.5 to about 24 hours or even longer, illustratively about 2 to about 24 hours, or about 2 to about 8 hours. In various embodiments, the morphine-containing composition has at least one non-lipoidal or hydrophilic internal phase and at least one lipoidal or hydrophobic external phase that is bioadhesive to the mucosal surface, as more fully described hereinbelow.

As in the case of the phenyloin-containing composition, of particular usefulness is a morphine-containing composition exhibiting both bioadherence to the oral mucosal surface and controlled release, more especially sustained release, of morphine from the composition to the mucosa. Slow and sustained release of morphine is believed to result in maintenance of a concentration of morphine effective for analgesia in the mucosal and submucosal tissues, whereas an immediate or bolus release can lead, at least temporarily, to excessively high concentrations in the tissues that can increase the tendency for systemic uptake, resulting in the morphine being removed from the local site where its effect is desired to other parts of the body where it may produce unwanted side effects. Systemic delivery of morphine may also be more likely than topical delivery to lead to addiction.

In one embodiment, the morphine is administered intraorally in a composition wherein bioadherence to the oral mucosal surface is sufficient to provide a retention period as described above, and wherein release of the morphine is sustained for most (i.e., more than about half) to all of the retention period. This can be accomplished, for example, by use of a composition having at least one non-lipoidal internal phase and at least one lipoidal external phase as described hereinbelow.

A single such composition comprising both phenyloin and morphine provides numerous advantages, for example in ease and convenience of use, over separate compositions. In one embodiment, a composition and dosage regimen are selected to promote topical delivery of phenyloin and morphine to the mucosal surface without substantial systemic absorption of phenyloin or morphine. In another embodiment, where at least some systemic delivery of morphine is desired, a composition and dosage regimen are selected to promote topical delivery of phenyloin to the mucosal surface without substantial systemic delivery of phenyloin, but to permit absorption of a therapeutically effective systemic level of morphine.

As an alternative to, or in addition to, morphine or other opioid as an analgesic agent to be administered topically in combination with phenyloin, a non-opioid analgesic such as benzydamine or capsaicin and/or a locally acting anesthetic such as lidocaine, lignocaine, benzocaine, xylocalne, dyclonine or diphenhydramine can be used.

A method of the invention for treating oral mucositis can optionally further comprise topically administering to the oral mucosal surface at least one additional therapeutic agent other than an analgesic agent.

In one embodiment, the additional therapeutic agent comprises a biologic response modifier, for example an immunomodulating agent such as keratinocyte growth factor (KGF), e.g., palifermin (a recombinant human KGF). Other biologic response modifiers that may be useful include granulocyte colony-stimulating factor (G-CSF), granulocyte-macrophage colony stimulating factor (GM-CSF), epidermal growth factor, transforming growth factor β₃ and interleukin-11. A cytokine modulator such as an IL-1 inhibitor, TNFα inhibitor or NO synthase stimulator can also be included.

In a further embodiment, the additional therapeutic agent comprises an antimicrobial, for example an antifungal, antibacterial, antiviral or combination thereof. Illustrative antimicrobials include acyclovir, amphotericin B, clindamycin, clotrimazole, fluconazole, nystatin, polymixin E, tobramycin and valacyclovir.

Antimicrobials can be useful in management of secondary infections arising from an ulcerated or otherwise damaged oral mucosa. Such secondary infections can be predominantly fungal, e.g., candida infection, bacterial, e.g., streptococcus infection, or viral, e.g., herpes simplex infection, or can involve a combination of any two or all three of these types of infection. In less severe cases of oral mucositis, promotion of mucosal regeneration by treatment with phenyloin according to the present invention may be sufficient to prevent or moderate a secondary infection and the use of antimicrobials may be unnecessary.

A method of the invention optionally comprises administration of a further agent, in addition to phenyloin and optionally an analgesic or anesthetic, having utility in treatment of mucositis, for example glutamine, allopurinol, N-acetylcysteine, etc.

In a second aspect of the invention, a therapeutic combination comprises phenyloin and an analgesic agent, each in a pharmaceutical composition adapted for topical administration to a biological surface, for example an oral mucosal surface, and bioadhesive thereto. The combination can take a form of separate compositions, respectively comprising the phenyloin and the analgesic agent, or of a single composition comprising both the phenyloin and the analgesic agent.

Biological surfaces to which the compositions useful in such a therapeutic combination can be bioadhesive include without limitation epidermal surfaces such as skin, for example at a locus or in the vicinity of a wound thereto, and mucosal surfaces such as oral, pharyngeal, nasal, esophageal, gastrointestinal, rectal and vaginal mucosa.

The analgesic agent can illustratively be selected from those mentioned above, comprising for example an opioid such as morphine and/or a pharmaceutically acceptable salt thereof.

In a third aspect of the invention, a pharmaceutical composition comprises phenyloin in an excipient vehicle suitable for intraoral administration. The composition according to this aspect is bioadhesive to biological surface, for example any surface as mentioned above, more particularly an oral mucosal surface.

In some important embodiments, the composition has at least one non-lipoidal internal phase and at least one lipoidal external phase that is bioadhesive to the oral mucosal surface. Such a composition has characteristics of a water-in-oil emulsion, as described for example in the patents individually cited below and incorporated by reference herein.

Above-cited U.S. Pat. No. 5,554,380.

Above-cited U.S. Pat. No. 5,858,391.

The active agent(s), i.e., phenyloin and optionally at least one additional active agent, for example an analgesic such as morphine, can each independently be present in either or both of the internal and external phases. Typically, active agents in insoluble form are located predominantly in the external hydrophobic phase, and active agents in more soluble form, for example phenyloin sodium or morphine sulfate, are located at least in part in the internal hydrophilic phase.

The composition can be in liquid, semi-solid or solid form. In some embodiments, the composition has a viscosity of about 20,000 to about 1,000,000 centipoise. If solid or semi-solid, the composition in some embodiments liquefies at body temperatures, aiding dispersal within the mouth cavity where it coats the oral mucosa and other surfaces. In some embodiments the emulsion system is delivered in a suitable pharmaceutical carrier, chewing gum composition, confectionery composition, or other orally acceptable carrier vehicle.

The emulsion system generates a protective film that can increase in thickness during the residence period by absorption of oral fluids into the hydrophilic layer of the system. This can then provide a thicker protective layer.

It is believed that the presence of one or more emulsifiers in the external phase are important to the bioadhesive property of the composition. The emulsifier(s) can also be important in preventing the internal phase, which is typically discontinuous, from coalescing, thus providing stability to the emulsion system before use and during the period of contact of the emulsion system with the oral mucosal surface. This stability, in addition to the bioadhesive nature of the external phase, is believed to provide sustained release properties in accordance with some embodiments of the present invention. Emulsifiers having an HLB value less than about 10 are generally preferred.

In a first illustrative embodiment of a water-in-oil emulsion composition, the internal hydrophilic (non-lipoidal) phase constitutes about 25% to about 75%, for example about 50% to about 75% or about 60% to about 75%, and the external hydrophobic (lipoidal) phase constitutes about 25% to about 75%, for example about 25% to about 50% or about 25% to about 40%, by volume of the emulsion system. Typically the volume of the internal phase is at least equal to that of the external phase. In some compositions, referred to herein as “high internal phase ratio” compositions, the internal phase is substantially greater in volume than the external phase, for example having an internal/external phase ratio of about 2:1 to about 3:1. The external hydrophobic phase according to the present embodiment comprises one or more emulsifiers in an amount of about 5% to about 50%, for example about 10% to about 30%, one or more glyceride esters of long chain (Cl₁₂₋₃₂) fatty acids in an amount of 0% to about 75%, for example about 20% to about 70%, and one or more wax materials having a melting point within the range of about 50° C. to about 200° C., in an amount of about 4% to about 50%, for example about 10% to about 40%, by weight of the hydrophobic phase. The internal hydrophilic phase typically comprises one or more ingredients selected from water, glycerin, sorbitol, sugars, water-soluble polymers and mixtures thereof.

The hydrophilic phase illustratively comprises sorbitol and water. The sorbitol can be supplied for example in a form of a sorbitol solution containing about 70% by weight sorbitol. Alternatively or in addition, the hydrophilic phase can comprise one or more sugars other than sorbitol, supplied for example in a form of a syrup such as corn syrup or high fructose corn syrup, or a sugar solution such as can sugar solution, dextrose solution, maltitol solution and the like. Optionally, the hydrophilic phase comprises one or more water-soluble polymers, for example polyethylene glycols.

The emulsion system contains and is at least partially stabilized by one or more emulsifiers. Typically, the emulsifiers are soluble in the external hydrophobic phase. Suitable emulsifiers are pharmaceutically acceptable oil-miscible surface-active compounds including, for example, fatty acid esters of glycerol (e.g., glyceryl monoisostearate), low molecular weight polyglycerols, mono- and diglyceride mixtures, propylene glycol diesters of medium-chain fatty acids (e.g., Miglyol™ 840 of Sasol), polyglycerol-3-oleate and the like, alone or with addition of a metallic soap such as aluminum stearate. Additional emulsifiers present in the hydrophobic phase can include sorbitan esters (e.g., sorbitan monooleate), lecithin (e.g., a soy lecithin product such as Phospholipon™ 90G), 1- to 5-mole ethoxylates of fatty acids or alcohols, dipolyhydroxystearate esters (e.g., PEG 30 dipolyhydroxystearate), saccharide derivatives and mixtures thereof.

It is believed that the one or more emulsifiers in the external phase are important to the bioadhesive property of the composition. The emulsifier(s) can also be important in preventing the internal phase, which is typically discontinuous, from coalescing, thus providing stability to the emulsion system before use and during the period of contact of the emulsion system with the oral mucosal surface. This stability, in addition to the bioadhesive nature of the external phase, is believed to provide sustained release properties in accordance with some embodiments of the present invention. Emulsifiers having an HLB value less than about 10 are generally preferred.

Glyceride esters included in the external phase typically comprise a blend of mono-, di-, and/or triglyceride esters of long chain fatty acids such as lauric, myristic, palmitic, stearic, oleic, linoleic and linolenic acids. It is generally preferred to select glyceride esters having melting points of about 30° C. to about 50° C.

Wax materials included in the external phase can be selected from animal waxes, vegetable waxes, petroleum waxes, synthetic waxes and mixtures thereof. Suitable waxes include without limitation beeswax, candelilla wax, carnauba wax, microcrystalline wax and mixtures thereof.

The external hydrophobic phase optionally includes additional components including diluents, such as vegetable or mineral oils, and binders such as ethylcellulose, sodium carboxymethyl cellulose, polyvinylpyrrolidone and the like.

In a second illustrative embodiment of a water-in-oil emulsion composition, the internal hydrophilic (non-lipoidal) phase constitutes about 75% to about 99%, for example about 80% to about 90%, and the external hydrophobic (lipoidal) phase constitutes about 1% to about 25%, for example about 10% to about 20%, by volume of the emulsion system. Such a composition is a high internal phase ratio composition, for example having an internal/external phase ratio of about 4:1 to about 9:1. The external hydrophobic phase according to the present embodiment comprises one or more emulsifiers in an amount of about 3% to about 97%, for example about 10% to about 80% or about 20% to about 60%, and one or more oils in an amount of about 3% to about 97%, for example about 20% to about 90% or about 40% to about 80%, by weight of the hydrophobic phase. The internal hydrophilic phase typically comprises one or more ingredients selected from water, glycerin, sorbitol, sugars, water-soluble polymers and mixtures thereof.

As in the previous embodiment, the hydrophilic phase illustratively comprises sorbitol and water. The sorbitol can be supplied for example in a form of a sorbitol solution containing about 70% by weight sorbitol. Alternatively or in addition, the hydrophilic phase can comprise one or more sugars other than sorbitol, supplied for example in a form of a syrup such as corn syrup or high fructose corn syrup, or a sugar solution such as can sugar solution, dextrose solution, maltitol solution and the like. Optionally, the hydrophilic phase comprises one or more water-soluble polymers, for example polyethylene glycols.

As in the previous embodiment, suitable emulsifiers for the external hydrophobic phase are pharmaceutically acceptable oil-miscible surface-active compounds including, for example, fatty acid esters of glycerol, low molecular weight polyglycerols, mono- and diglyceride mixtures, propylene glycol diesters of medium-chain fatty acids, polyglycerol-3-oleate and the like, alone or with addition of a metallic soap such as aluminum stearate. Additional emulsifiers present in the hydrophobic phase can include sorbitan esters, lecithin, 1- to 5-mole ethoxylates of fatty acids or alcohols, dipolyhydroxystearate esters, saccharide derivatives and mixtures thereof.

Oil(s) for the external hydrophobic phase can be selected from a wide variety of materials, including mineral oils, vegetable oils, long chain (C₁₂₋₃₂), typically straight chain fatty acids, alcohols and esters and mixtures thereof.

Illustrative pharmaceutically acceptable oils include peanut oil, safflower oil, sunflower oil, soya oil, coconut oil, cottonseed oil, corn oil, olive oil, sesame oil, almond oil, castor oil, mineral oil (e.g., light mineral oil), isopropyl myristate and mixtures thereof.

Glyceride esters optionally included in the external phase can comprise a blend of mono-, di-, and/or triglyceride esters of long chain fatty acids such as lauric, myristic, palmitic, stearic, oleic, linoleic and linolenic acids.

Wax materials optionally included in the external phase can be selected from animal waxes, vegetable waxes, petroleum waxes, synthetic waxes and mixtures thereof. Suitable waxes include without limitation beeswax, candelilla wax, carnauba wax, microcrystalline wax and mixtures thereof.

A water-in-oil emulsion system as described herein can be prepared by continuous or batch processes. As in preparing conventional emulsions, shear force is applied to the system components, for example by use of homogenizers, mills, impingement surfaces, ultra-sound, shaking or vibration. Unlike conventional emulsions, the mixing shear should be at a relatively low level in order to prevent destruction of the system by imparting excess energy. Temperature is not usually a critical factor in preparation of the water-in-oil emulsion system. Temperatures utilized can depend upon the final end product desired.

Generally, the emulsions are prepared by separately making the hydrophilic and hydrophobic phases and then blending the phases together by adding the hydrophilic phase to the hydrophobic phase. The hydrophilic phase is then added in incremental amounts to the hydrophobic phase while mixing the components. As more and more of the hydrophilic phase is added the product begins to thicken. When the mixing is complete, the product can be pumped into a receiving vessel for final packaging or further processing.

In one embodiment, the active agent(s) and ingredients of the internal phase are mixed together at room temperature (about 24° C.). The ingredients of the external phase are mixed together in a separate vessel. The internal phase composition is slowly added to the external phase composition as the two phases are mixed together at low shear until the desired viscosity is obtained.

The internal phase can be mixed with the external phase in any suitable mixing device, for example a planetary-type mixer or a continuous mixer having multiple impellers. In the latter case, the external phase is first introduced into the continuous mixer until it reaches the level of the lowest impeller in the mixing chamber. The two phases are then simultaneously introduced through the bottom of the mixer in proper proportion as its impeller or impellers rotate to apply a shear to the components. The finished product emerges through the top of the mixer. The actual speed of the impeller or impellers can vary, as can the rate of flow of the two phase streams, depending upon the product to be produced.

The emulsion once prepared may be stored for future use, or formulated with one or more pharmaceutically acceptable excipients and/or carriers, to prepare pharmaceutical compositions which offer a variety of textures to suit particular dosage forms. Such compositions can be in the form of a lozenge, tablet, toffee, nougat, chewy candy, oral hygiene preparation, breath freshener, chewing gum or other oral formulation.

Preparation of Medicated Confectionery and Chewing Gum Products is Known in the art. When used in such delivery systems, the formulations of this invention may be blended with the confectionery or chewing gum product, coated on the surface thereof or center filled to enable the active agent(s) to be intraorally administered.

As used herein, the term “confectionery” relates to a product containing a bulking agent selected from sugars, syrups and sugar alcohols such as sorbitol or mannitol. In general, the bulking agent comprises about 5% to about 99%, for example about 20% to about 95%, by weight of the medicated confectionery product. Confectionery compositions include without limitation lozenges, tablets, toffees, nougats, chewy candies and the like.

Lozenges are flavored medicated dosage forms intended to be sucked and held in the mouth. They can take various shapes, the most common being flat, circular, octagonal and biconvex. Lozenge bases are generally of two forms: hard boiled candy lozenges and compressed tablet lozenges.

Hard boiled candy lozenges are prepared from a mixture of sugar and other carbohydrates that are kept in an amorphous or glassy condition. This form can be considered a solid syrup of sugars generally having about 0.1% to about 5%, for example about 0.5% to about 1.5%, moisture content. Such lozenges normally contain up to about 92% corn syrup or up to about 70% sugar, and are generally prepared from corn syrups high in dextrose, but can include other materials. Further ingredients such as flavorings, sweeteners, acidulants, colorants and so forth can also be added.

Alternatively, hard boiled candy lozenges can be prepared from nonfermentable sugars such as sorbitol, mannitol and hydrogenated corn syrup. Illustratively, such lozenges can contain up to about 95% sorbitol or a mixture of sorbitol and mannitol at a ratio of about 3:1 to about 19:1, and hydrogenated corn syrup up to about 55% of the syrup component.

Compressed tablet lozenges are formed into structures under pressure. They generally contain sugars in amounts up to 95% and typical tablet excipients such as binders and lubricants as well as flavors, colorants and the like.

A water-in-oil emulsion formulation of the invention is incorporated into the lozenge during mixing of ingredients.

Compositions can also be made of soft confectionery materials such as those contained in nougat. These materials contain two primary components, namely a high boiling syrup or “bob syrup” such as corn syrup or the like, and a relatively light textured aerated frappe, generally prepared from gelatin, egg albumen, milk proteins such as casein, and vegetable proteins such as soy protein or the like. The frappe is generally relatively light, having a density, for example, of about 0.5 to about 0.7 g/ml.

By comparison, the bob syrup is relatively viscous and possesses a higher density and frequently contains a substantial amount of sugar. Conventionally, the nougat composition is prepared by the addition of the bob syrup to the frappe under agitation, to form the basic nougat mixture. Further ingredients such as flavorings, oils, additional sugar and the like can be added thereafter, also under agitation, to provide a nougat candy base. A general discussion of the composition and preparation of nougat confections is found in Minifie (1980) Chocolate, Cocoa and Confectionery: Science and Technology, 2nd ed., AVI Publishing Co., Inc., Westport, Conn., pp. 424-425, the disclosure of which is incorporated herein by reference.

A water-in-oil emulsion of the invention can be admixed with a basic nougat mixture or nougat candy base, to form a homogenous mixture which can then be formed into suitable shapes.

Any conventional chewing gum composition can be used as a delivery vehicle for a water-in-oil emulsion of the invention. Without being limited to specific chewing gum formulations, illustrative examples are described in the patents individually cited below and incorporated herein by reference.

U.S. Pat. No. 4,683,138.

U.S. Pat. No. 4,775,537.

Chewing gums generally contain a gum base and modifiers to provide an acceptable texture and sweetness.

The gum base can contain conventional elastomer solvents to aid in softening the gum component. Such elastomer solvents can comprise methyl, glycerol or pentaerythritol esters of rosins or modified rosins, such as hydrogenated, dimerized or polymerized rosins or mixtures thereof. Examples of elastomer solvents suitable for use herein include pentaerythritol ester of partially hydrogenated wood or gum rosin, pentaerythritol ester of wood or gum rosin, glycerol ester of partially dimerized rosin, glycerol ester of polymerized rosin, glycerol ester of tall oil rosin, glycerol ester of wood or gum rosin and partially hydrogenated wood or gum rosin, partially hydrogenated methyl ester of rosin, and mixtures thereof. The elastomer solvent can be employed in an amount of about 10% to about 75%, for example about 45% to about 70%, by weight of the gum base.

A variety of traditional ingredients used as plasticizers or softeners such as lanolin, stearic acid, sodium stearate, potassium stearate, glycerol triacetate, glycerin, lecithin, glycerol monostearate and the like, can also be incorporated into the gum base to obtain a variety of desirable textures and consistency properties. These additional ingredients are generally employed in amounts up to about 30% by weight, most typically about 3% to about 5% by weight of the gum base.

Chewing gums can further comprise one or more sweetening agents (sweeteners). Sweetening agents can be selected from a wide range of materials including water-soluble natural and artificial sweeteners, dipeptide based sweeteners, protein based sweeteners and mixtures thereof.

Chewing gums optionally comprise conventional coloring agents such as titanium dioxide; emulsifiers such as lecithin or glyceryl monostearate; maltodextrins; and fillers such as aluminum hydroxide, alumina, aluminum silicates, talc, dicalcium phosphate, calcium carbonate, and combinations thereof. Fillers are typically present at up to about 25% by weight of the gum base.

Chewing gum compositions can be produced by techniques well known to those skilled in the art. For example, using conventional equipment the gum base is heated to temperatures sufficiently high enough to soften the base without adversely affecting the physical and chemical constitution of the base. The optimum temperature utilized can vary depending on the composition of the gum base used, but such temperatures are readily determined by those skilled in the art without undue experimentation. For example, suitable temperatures for softening the gum base are typically about 70° C. to about 90° C. Temperatures of about 40° C. to about 60° C. may be used with the gum base compositions disclosed in, for example, U.S. Pat. No. 4,587,125. During heating, the gum base is mixed with any of the optional components traditionally used with the gum base, such as plasticizers and elastomer solvents. In general, the order of addition of the various ingredients of the chewing gum composition is not critical. Flavoring agents, however, should be added when the gum base has been allowed to cool to a temperature below the volatilization temperature of the flavoring agents used. Flavoring agents can be added separately or blended together as a preblend before their addition. The mixture so produced is then extruded, using conventional equipment, and formed into suitable chewing gum shapes. A water-in-oil emulsion of the invention can be added during formation of the gum product or after it is formed, for example before or after addition of flavoring agents.

To prepare a center filled gum, a water-in-oil emulsion of the invention can be forced into the lumen of a hollow-centered rope of chewing gum, which is then pinched or otherwise segmented to form individual pieces. The emulsion is released into the oral cavity upon chewing the gum composition.

Flavoring agents are optionally present in the water-in-oil emulsion or in a delivery system such as a confectionery or chewing gum composition incorporating the emulsion. Natural and/or synthetic flavoring agents can be used, for example to enhance or mask an unpleasant taste. Representative flavoring, taste-enhancing or taste-masking agents include without limitation spearmint oil, peppermint oil, cinnamon oil, oil of wintergreen, citrus oils such as lemon, orange, grape, lime and grapefruit oils, and fruit essences such as apple, strawberry, cherry and pineapple essences, and pharmaceutically acceptable synthetic imitations thereof.

The amount of flavoring agent employed is normally a matter of preference subject to such factors as flavor type, composition type and strength desired. In general, amounts of about 0.05% to about 25%, for example about 0.3% to about 10% or about 0.8% to about 8% by weight of the final product are useful.

Sweetening agents are optionally present in the water-in-oil emulsion or in a delivery system such as a confectionery or chewing gum composition incorporating the emulsion. Natural and/or synthetic flavoring agents can be used. Representative sweetening agents include without limitation:
water-soluble monosaccharides, disaccharides and polysaccharides such as xylose, ribose, glucose, mannose, galactose, fructose, dextrose, sucrose, maltose, partially hydrolyzed starch, or corn syrup solids; sugar alcohols such as sorbitol, xylitol or mannitol; and mixtures thereof;
water-soluble artificial sweeteners such as salts of saccharin, cyclamic acid or acesulfame; saccharin free acid; and mixtures thereof;
dipeptide based sweeteners such as L-aspartyl-L-phenylalanine methyl ester and materials described, for example, in U.S. Pat. No. 3,492,131;

Representative examples of other conventional additives include without limitation:
preservatives such as benzoic acid, sorbic acid, methylparaben, propylparaben, ethylenediaminetetraacetic acid (EDTA) and mixtures thereof, typically present in a total amount of 0% to about 1%, for example about 0.05% to about 0.5% by weight of the composition;
buffers such as citric acid-sodium citrate, phosphoric acid-sodium phosphate, and acetic acid-sodium acetate, typically present in a total amount of 0% to about 1%, for example about 0.05% to about 0.5% by weight of the composition;
suspending agents or thickeners including cellulosics (e.g., methylcellulose, hydroxypropylcellulose, hydroxypropylmethylcellulose, etc.), carrageenans, alginic acid and derivatives thereof, xanthan gums, gelatin, acacia, microcrystalline cellulose and mixtures thereof, typically present in a total amount of 0% to about 20%, for example about 1% to about 15% by weight of the composition;
antifoaming agents such as dimethylpolysiloxane, typically present in a total amount of 0% to about 0.2%, for example about 0.01% to about 0.1% by weight of the composition;
colorants including pigments such as titanium dioxide, typically present in a total amount of up to about 1%, and dyes such as FD&C dyes approved for food, drug and cosmetic applications, typically present in a total amount of 0% to about 0.25%, for example about 0.05% to about 0.2% by weight of the composition;
decolorizing agents such as sodium metabisulfite, ascorbic acid and the like, included to inhibit color changes due to aging, typically present in a total amount of 0% to about 0.25%, for example about 0.05% to about 0.2% by weight of the composition;
solubilizers such as alcohol, propylene glycol, polyethylene glycol and the like, included for example to solubilize flavoring agents, typically present in a total amount of 0% to about 10%, for example about 2% to about 5% by weight of the composition.

A water-in-oil emulsion of the present invention can be employed, with or without conventional supplemental agents, as a principal component of a system to be dissolved or dispersed in water or other ingestible liquid to form, for example, a mouthwash, oral rinse, oropharyngeal spray liquid or swish and swallow liquid.

Alternatively, a water-in-oil emulsion of the present invention can be employed as a component of a conventionally prepared paste, ointment, gel or dentifrice for intraoral administration.

As indicated above, in a low-volume (about 0.5 to about 2 ml per dose) composition, concentration of phenyloin can illustratively be about 10 to about 400 mg/ml; in a medium-volume (about 2 ml to about 15 ml per dose) composition, illustratively about 1 to about 100 mg/ml; and in a high-volume (about 15 ml to about 25 ml per dose) composition, illustratively about 1 to about 30 mg/ml.

Optionally, the composition further comprises a second agent that can co-act with the phenyloin to inhibit mucosal degeneration and/or promote mucosal regeneration. Alternatively or in addition, the second agent can have a palliative effect and/or can treat a secondary condition accompanying the oral mucositis, for example a microbial infection. As mentioned above, such a second agent can comprise a therapeutic agent and/or a nutritional agent such as vitamin E, vitamin C, vitamin A or glutamine. A therapeutic agent useful in combination with phenyloin can illustratively be selected from analgesics, biologic response modifiers, antihistaminics, antimicrobials and antiseptics. Combinations of two or more agents from one or more of these classes can also be useful.

A presently preferred composition of the invention comprises (a) phenyloin in an amount effective, upon topical administration of the composition to an oral mucosal surface of a subject having oral mucositis, to inhibit mucosal degeneration or promote mucosal regeneration, and (b) an analgesic agent in an amount effective, in combination with the phenyloin upon topical administration of the composition to an oral mucosal surface, to reduce pain associated with oral mucositis.

The analgesic agent can be selected from anesthetics, opioids and non-opioid analgesics. Combinations of two or more agents from one or more of these classes can also be useful.

In a more particular embodiment, the analgesic agent comprises an opioid, illustratively comprising at least one agent selected from the group listed hereinabove. In one embodiment, the opioid comprises morphine and/or a pharmaceutically acceptable salt thereof, for example morphine sulfate or morphine hydrochloride.

In a low-volume (about 0.5 to about 2 ml per dose) composition, concentration of morphine can illustratively be about 5 to about 200 mg/ml; in a medium-volume (about 2 ml to about 15 ml per dose) composition, illustratively about 1 to about 50 mg/ml; and in a high-volume (about 15 ml to about 25 ml per dose) composition, illustratively about 0.5 to about 10 mg/ml.

As an alternative to, or in addition to, morphine or other opioid as an analgesic agent to be included with phenyloin in a composition of the invention, a non-opioid analgesic such as benzydamine or capsaicin and/or a locally acting anesthetic such as lidocaine, lignocaine, benzocaine, xylocaine, dyclonine or diphenhydramine can be used.

The additional therapeutic agent, if present in the composition with phenyloin, can alternatively or additionally comprise a biologic response modifier, for example an immunomodulating agent such as keratinocyte growth factor (KGF), e.g., palifermin (a recombinant human KGF). Other biologic response modifiers that may be useful include granulocyte colony-stimulating factor (G-CSF), granulocyte-macrophage colony stimulating factor (GM-CSF), epidermal growth factor, transforming growth factor β₃ and interleukin-11. A cytokine modulator such as an IL-1 inhibitor, TNFα inhibitor or NO synthase stimulator can also be included.

The additional therapeutic agent, if present in the composition with phenyloin, can alternatively or additionally comprise an antimicrobial, for example an antifungal, antibacterial, antiviral or combination thereof. Illustrative antimicrobials include acyclovir, amphotericin B, clindamycin, clotrimazole, fluconazole, nystatin, polymixin E, tobramycin and valacyclovir.

A composition of the invention optionally comprises a further agent, in addition to phenyloin and optionally an analgesic or anesthetic, having utility in treatment of mucositis, for example glutamine, allopurinol, N-acetylcysteine, etc.

Illustratively, the following ingredients can be used in preparing oral emulsion compositions of the invention (Formulations I-VII below).

Formulation I comprises phenyloin, morphine sulfate, water, glycerin, a pH buffer (e.g., acetic acid and sodium acetate), light mineral oil, polyglycerol-3-oleate, sodium chloride, sucrose, one or more preservatives (e.g., methyl-, propyl- and butylparabens) and, optionally, flavoring (e.g., peppermint). The emulsion is viscous and bioadhesive. When administered it coats the inside of the mouth, remains adhered to the oral mucosa for one to several hours and delivers the active ingredients locally during that period.

Formulation II comprises phenyloin, morphine sulfate, water, a pH buffer, sorbitol, light mineral oil, one or more surfactants, one or more salts, one or more sugars, one or more preservatives and, optionally, flavoring. The emulsion is viscous and bioadhesive. When administered it coats the inside of the mouth, remains adhered to the oral mucosa for a period of time and delivers the active ingredients locally during that period.

Formulation III comprises phenyloin, morphine sulfate, water, EDTA, sorbitol, light mineral oil, surfactants (Phospholipon™ 90G and polyglycerol-3-oleate), microcrystalline wax, silica, one or more salts, sugar, one or more preservatives and, optionally, flavoring. The emulsion is moderately viscous and bioadhesive. When administered it coats the inside of the mouth, remains adhered to the oral mucosa for a period of time and delivers the active ingredients locally during that period.

Formulation IV comprises phenyloin, morphine sulfate, water, EDTA, sorbitol, light mineral oil, surfactants (PEG 30 dipolyhydroxystearate and polyglycerol-3-oleate), microcrystalline wax, silica, one or more salts, sugar, one or more preservatives and, optionally, flavoring. The emulsion is moderately viscous and bioadhesive. When administered it coats the inside of the mouth, remains adhered to the oral mucosa for a period of time and delivers the active ingredients locally during that period.

Formulation V comprises phenyloin, morphine sulfate, water, EDTA, sorbitol, mineral oil, surfactants (PEG 30 dipolyhydroxystearate, polyglycerol-3-oleate and glyceryl monoisostearate), microcrystalline wax, silica, one or more salts, sugar, one or more preservatives and, optionally, flavoring. The emulsion is moderately viscous and bioadhesive. When administered it coats the inside of the mouth, remains adhered to the oral mucosa for a period of time and delivers the active ingredients locally during that period.

Formulation VI comprises phenyloin, morphine sulfate, water, EDTA, sorbitol, mineral oil, surfactants (Phospholipon™ 90G, polyglycerol-3-oleate and glyceryl monoisostearate), microcrystalline wax, silica, one or more salts, sugar, one or more preservatives and, optionally, flavoring. The emulsion is moderately viscous and bioadhesive. When administered it coats the inside of the mouth, remains adhered to the oral mucosa for a period of time and delivers the active ingredients locally during that period.

Formulation VII comprises phenyloin, morphine sulfate, water, EDTA, sorbitol, pH buffer, vegetable oil (e.g., sunflower oil), surfactants (soy lecithin, sorbitan monooleate and Miglyol™ 840), microcrystalline wax, silica, one or more salts, sugar, one or more preservatives and, optionally, flavoring. The emulsion is viscous and bioadhesive. When administered it coats the inside of the mouth, remains adhered to the oral mucosa for a period of time and delivers the active ingredients locally during that period.

In a fourth aspect of the invention, a method for relieving pain from oral mucositis associated with cancer therapy in a subject comprises administering to an oral mucosal surface of the subject a pharmaceutical composition comprising an analgesic agent that comprises morphine and/or a pharmaceutically acceptable salt thereof in an excipient vehicle suitable for intraoral administration, the composition having at least one non-lipoidal internal phase and at least one lipoidal external phase that is bioadhesive to the oral mucosal surface.

Any morphine-containing composition meeting the above criteria can be used, including compositions described hereinabove for use in combination with phenyloin. Doses of morphine, retention time on the oral mucosal surface, release properties and other details of administration, including dosage frequency, can also be as described above for compositions described hereinabove.

In a fifth aspect of the invention, a cancer treatment regimen comprises (a) administering to a cancer patient radiotherapy and/or chemotherapy at a level sufficient to result in oral mucositis, and (b) topically administering phenyloin or a pharmaceutically effective salt thereof to an oral mucosal surface of the patient in an amount effective to inhibit mucosal degeneration or promote mucosal regeneration.

The phenyloin can be administered prophylactically, before appearance of symptoms of oral mucositis. Alternatively or in addition, the phenyloin can be administered after appearance of oral mucositis.

Optionally, such a regimen, further comprises (c) topically administering to the oral mucosal surface an analgesic agent, comprising for example an opioid such as morphine and/or a pharmaceutically acceptable salt thereof, in an amount effective, in combination with the phenyloin or salt thereof, to reduce pain associated with the oral mucositis. Typically the analgesic agent is not used prophylactically, but on development of oral mucositis, especially painful or severe oral mucositis.

In a sixth aspect of the invention, a cancer treatment regimen comprises administering to a cancer patient radiotherapy and/or chemotherapy at a level sufficient to result in oral mucositis, and topically administering to an oral mucosal surface a pharmaceutical composition that comprises an analgesic agent comprising morphine and/or a pharmaceutically acceptable salt thereof, in an excipient vehicle suitable for intraoral administration, the composition having at least one non-lipoidal internal phase and at least one lipoidal external phase that is bioadhesive to the mucosal surface.

According to the above fifth and sixth aspects of the invention, a regimen as described is of particular use and value in a situation wherein tolerance of the patient for the radiotherapy and/or chemotherapy is limited at least in part by severity of oral mucositis occurring as an adverse side effect of the radiotherapy and/or chemotherapy. In such a situation, reduction of severity of the oral mucositis resulting from said topical administration of phenyloin or salt thereof can permit more aggressive treatment of the patient's cancer. This more aggressive treatment, in turn, can substantially improve the outcome of the regimen, for example by reducing morbidity of the cancer, by increasing the probability of eradication of the cancer, by lengthening a period of remission, and/or by prolonging life.

In some embodiments, a regimen according to either of the above aspects comprises chemotherapy. The oral mucositis can result from a systemic effect of such chemotherapy.

In other embodiments, a regimen according to either of the above aspects comprises radiation therapy. The oral mucositis can result from a local effect of such radiation therapy, particularly where the cancer being treated by the radiation is a head and neck cancer.

In a seventh aspect of the invention, a method for treating a painful skin lesion comprises topically administering to the lesion and/or an area of skin adjacent thereto a composition comprising phenyloin or a pharmaceutically acceptable salt thereof in an amount effective to promote healing of the lesion, said composition having at least one non-lipoidal internal phase and at least one lipoidal external phase that is bioadhesive to a skin surface.

Optionally, the composition administered according to such a method further comprises an analgesic agent, comprising for example an opioid such as morphine and/or a pharmaceutically acceptable salt thereof, in an amount effective to reduce pain associated with the lesion.

Any composition meeting the above criteria can be used, including compositions comprising phenyloin alone or in combination with an analgesic agent as described hereinabove. The composition can be applied occlusively, for example with a sterile dressing, or non-occlusively.

The method can be used to treat a variety of skin lesions, including without limitation puncture and abrasion wounds, for example surgical and non-surgical (e.g., sustained through accident, violence or war) wounds, burns and ulcers, for example decubitus ulcers.

All patents and publications cited herein are incorporated by reference into this application in their entirety.

The words “comprise”, “comprises”, and “comprising” are to be interpreted inclusively rather than exclusively.

What is claimed is:

1. A method for treating oral mucositis in a subject, comprising topically administering phenyloin or a pharmaceutically acceptable salt thereof to an oral mucosal surface of the subject in an amount effective to inhibit mucosal degeneration or promote mucosal regeneration.

2. The method of claim 1, wherein the subject has received, is receiving or later receives cancer therapy comprising chemotherapy, radiation therapy or a combination thereof, said cancer therapy being associated with risk or incidence of oral mucositis.

3. The method of claim 1, wherein the phenyloin or salt thereof is administered at a dose of about 5 to about 500 mg/day.

4. The method of claim 1, wherein the phenyloin or salt thereof is administered at a dose of about 25 to about 200 mg/day.

5. The method of claim 1, wherein the phenyloin is administered as the sodium salt thereof.

6. The method of claim 1, wherein the phenyloin or salt thereof is administered in a pharmaceutical composition adapted for intraoral administration.

7. The method of claim 6, wherein the composition is administered intraorally in an amount of about 0.5 to about 25 ml.

8. The method of claim 6, wherein the composition is administered intraorally in an amount of about 2 ml to about 15 ml.

9. The method of claim 1, further comprising topically administering to the oral mucosal surface an additional agent selected from the group consisting of analgesics, biologic response modifiers, antihistaminics, antimicrobials, antiseptics, nutritional agents and combinations thereof.

10. The method of claim 9, wherein the additional agent is an analgesic agent and is administered in an amount effective, in combination with the phenyloin or salt thereof, to reduce pain associated with the oral mucositis.

11. The method of claim 10, wherein the analgesic agent is selected from the group consisting of anesthetics, opioids and non-opioid analgesics.

12. The method of claim 10, wherein the analgesic agent comprises an opioid.

13. The method of claim 12, wherein the opioid comprises at least one agent selected from the group consisting of alfentanil, alphaprodine, anileridine, benzylmorphine, bezitramide, buprenorphine, butorphanol, clonitazene, codeine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, hydrocodone, hydromorphone, hydroxypethidine, isomethadone, ketobemidone, levorphanol, lofentanil, meperidine, meptazinol, metazocine, methadone, metopon, morphine, myrophine, nalbuphine, narceine, nicomorphine, norlevorphanol, normethadone, normorphine, norpipanone, opium, oxycodone, oxymorphone, papaveretum, pentazocine, phenadoxone, phenazocine, phenoperidine, piminodine, piritramide, proheptazine, promedol, propiram, propoxyphene, remifentanil, sufentanil, tilidine, and pharmaceutically acceptable salts, esters, prodrugs and enantiomers thereof.

14. The method of claim 12, wherein the opioid comprises morphine and/or a pharmaceutically acceptable salt thereof.

15. The method of claim 14, wherein the morphine is administered as morphine sulfate.

16. The method of claim 14, wherein the morphine or salt thereof is administered at a dose of about 5 to about 200 mg/day.

17. The method of claim 14, wherein the morphine or salt thereof is administered at a dose of about 10 to about 100 mg/day.

18. The method of claim 10, wherein the analgesic agent is administered in a pharmaceutical composition adapted for intraoral administration.

19. The method of claim 18, wherein the analgesic agent and the phenyloin or salt thereof are administered in separate pharmaceutical compositions at the same or different times.

20. The method of claim 18, wherein the analgesic agent and the phenyloin or salt thereof are administered together in a single pharmaceutical composition.

21. The method of claim 20, wherein the composition, phenyloin dose and analgesic dose are selected to promote topical delivery of phenyloin and analgesic agent to the mucosal surface without substantial systemic absorption of phenyloin or analgesic agent.

22. The method of claim 20, wherein the composition, phenyloin dose and analgesic dose are selected to promote topical delivery of phenyloin to the mucosal surface without substantial systemic delivery of phenyloin, but to permit absorption of a therapeutically effective systemic level of the analgesic agent.

23. The method of claim 20, wherein the composition comprises a mouthwash, swish and swallow liquid, viscous liquid, gel, ointment, paste, powder, reconstituted powder, film, chew, lozenge, troche, candy, lollipop, popsicle, chewing gum, sublingual or buccal tablet, quick-dissolve or quick-melt tablet, medicated wad or dressing, dentifrice or oropharyngeal spray.

24. The method of claim 20, wherein the composition is bioadhesive to the mucosal surface.

25. The method of claim 24, wherein the composition has at least one non-lipoidal internal phase and at least one lipoidal external phase that is bioadhesive to the mucosal surface.

26. The method of claim 24, wherein the composition is sufficiently bioadhesive to exhibit retention on the mucosal surface for a period of about 0.5 to about 24 hours.

27. The method of claim 26, wherein release of the phenyloin and/or the analgesic agent from the composition to the mucosal surface is sustained for most to all of the retention period.

28. The method of claim 26, wherein the composition is administered 1 to about 8 times per day.

29. The method of claim 26, wherein the composition is administered 1 to about 6 times per day.

30. The method of claim 26, wherein the composition is administered 1 to 2 times per day.

31. A therapeutic combination comprising phenyloin and an analgesic agent, each in a pharmaceutical composition adapted for topical administration to a biological surface and bioadhesive thereto.

32. The combination of claim 31, wherein each composition is adapted for intraoral administration and is bioadhesive to an oral mucosal surface.

33. A pharmaceutical composition comprising phenyloin or a pharmaceutically acceptable salt thereof in an excipient vehicle suitable for intraoral administration, said composition being bioadhesive to an oral mucosal surface.

34. The composition of claim 33, having at least one non-lipoidal internal phase and at least one lipoidal external phase that is bioadhesive to the oral mucosal surface.

35. The composition of claim 33, wherein the phenyloin or salt thereof is present at a concentration of about 1 to about 400 mg/ml.

36. The composition of claim 33, wherein the phenyloin or salt thereof is present at a concentration of about 1 to about 100 mg/ml.

37. The composition of claim 33, further comprising an analgesic agent.

38. The composition of claim 37, wherein the analgesic agent is selected from the group consisting of anesthetics, opioids and non-opioid analgesics.

39. The composition of claim 37, wherein the analgesic agent comprises an opioid.

40. The composition of claim 39, wherein the opioid comprises at least one agent selected from the group consisting of alfentanil, alphaprodine, anileridine, benzylmorphine, bezitramide, buprenorphine, butorphanol, clonitazene, codeine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, hydrocodone, hydromorphone, hydroxypethidine, isomethadone, ketobemidone, levorphanol, lofentanil, meperidine, meptazinol, metazocine, methadone, metopon, morphine, myrophine, nalbuphine, narceine, nicomorphine, norlevorphanol, normethadone, normorphine, norpipanone, opium, oxycodone, oxymorphone, papaveretum, pentazocine, phenadoxone, phenazocine, phenoperidine, piminodine, piritramide, proheptazine, promedol, propiram, propoxyphene, remifentanil, sufentanil, tilidine, and pharmaceutically acceptable salts, esters, prodrugs and enantiomers thereof.

41. The composition of claim 39, wherein the analgesic agent comprises morphine and/or a pharmaceutically acceptable salt thereof.

42. The composition of claim 41, wherein the analgesic agent comprises morphine sulfate.

43. The composition of claim 41, wherein the morphine or salt thereof is present at a concentration of about 1 to about 200 mg/ml.

44. The composition of claim 41, wherein the morphine or salt thereof is present at a concentration of about 1 to about 20 mg/ml.

45. The composition of claim 33, that is in a form of a mouthwash, swish and swallow liquid, viscous liquid, gel, ointment, paste, powder, reconstituted powder, film, chew, lozenge, troche, candy, lollipop, popsicle, chewing gum, sublingual or buccal tablet, quick-dissolve or quick-melt tablet, medicated wad or dressing, dentifrice or oropharyngeal spray.

46. A method for relieving pain from oral mucositis associated with cancer therapy in a subject, comprising administering to an oral mucosal surface of the subject a pharmaceutical composition comprising an analgesic agent that comprises morphine and/or a pharmaceutically acceptable salt thereof in an excipient vehicle suitable for intraoral administration, said composition having at least one non-lipoidal internal phase and at least one lipoidal external phase that is bioadhesive to an oral mucosal surface.

47. The method of claim 46, wherein the analgesic agent comprises morphine sulfate.

48. The method of claim 47, wherein the morphine or salt thereof is present at a concentration of about 0.5 to about 200 mg/ml.

49. The method of claim 47, wherein the morphine or salt thereof is present in the composition at a concentration of about 1 to about 50 mg/ml.

50. The method of claim 46, wherein the composition is administered in an amount and frequency providing a morphine dose of about 5 to about 200 mg/day.

51. The method of claim 46, wherein the composition is administered in an amount and frequency providing a morphine dose of about 10 to about 100 mg/day.

52. A cancer treatment regimen comprising administering to a cancer patient radiotherapy and/or chemotherapy at a level sufficient to result in oral mucositis, and topically administering phenyloin or a pharmaceutically effective salt thereof to an oral mucosal surface of the patient in an amount effective to inhibit mucosal degeneration or promote mucosal regeneration.

53. The regimen of claim 52, further comprising topically administering to the oral mucosal surface an analgesic agent in an amount effective to reduce pain associated with oral mucositis.

54. The regimen of claim 52, wherein the phenyloin or salt thereof is administered prophylactically prior to appearance of oral mucositis.

55. The regimen of claim 52, wherein the phenyloin or salt thereof is administered after appearance of oral mucositis.

56. The regimen of claim 52, wherein tolerance of the patient for the radiotherapy and/or chemotherapy is limited at least in part by severity of oral mucositis occurring as an adverse side effect of the radiotherapy and/or chemotherapy; and wherein reduction of severity of the oral mucositis resulting from said topical administration of phenyloin or salt thereof permits more aggressive treatment of the patient's cancer.

57. The regimen of claim 52, wherein the oral mucositis results from a systemic effect of chemotherapy.

58. The regimen of claim 52, wherein the oral mucositis results from a local effect of radiation therapy.

59. The regimen of claim 58, wherein the cancer being treated is a head and neck cancer.

60. A cancer treatment regimen comprising administering to a cancer patient radiotherapy and/or chemotherapy at a level sufficient to result in oral mucositis, and topically administering to an oral mucosal surface a pharmaceutical composition that comprises an analgesic agent comprising morphine and/or a pharmaceutically acceptable salt thereof in an excipient vehicle suitable for intraoral administration, said composition having at least one non-lipoidal internal phase and at least one lipoidal external phase that is bioadhesive to the mucosal surface.

61. The regimen of claim 60, wherein the analgesic agent comprises morphine sulfate.

62. The regimen of claim 60, wherein tolerance of the patient for the radiotherapy and/or chemotherapy is limited at least in part by severity of oral mucositis occurring as an adverse side effect of the radiotherapy and/or chemotherapy; and wherein reduction of pain associated with the oral mucositis resulting from said topical administration of said composition permits more aggressive treatment of the patient's cancer.

63. The regimen of claim 60, wherein the oral mucositis results from a systemic effect of chemotherapy.

64. The regimen of claim 60, wherein the oral mucositis results from a local effect of radiation therapy.

65. The regimen of claim 64, wherein the cancer being treated is a head and neck cancer.

66. A method for treating a painful skin lesion, comprising topically administering to the lesion and/or an area of skin adjacent thereto a composition comprising phenyloin or a pharmaceutically acceptable salt thereof in an amount effective to promote healing of the lesion, said composition having at least one non-lipoidal internal phase and at least one lipoidal external phase that is bioadhesive to a skin surface.

67. The method of claim 66, wherein the composition further comprises an analgesic agent in an amount effective to reduce pain associated with the lesion.

68. The method of claim 67, wherein the analgesic agent comprises an opioid.

69. The method of claim 67, wherein the analgesic agent comprises morphine and/or a pharmaceutically acceptable salt thereof.

70. The method of claim 67, wherein the analgesic agent comprises morphine sulfate.

71. The method of claim 66, wherein the skin lesion comprises a surgical or non-surgical wound, a burn or an ulcer.
